(19) United States
(12) Patent Application Publication
(10) Pub. No.: US 2002/0165456 A1
(43) Pub. Date: Nov. 7, 2002

(54) Estimation of the average size of white light scatterers in normal and cancerous tissue using light scattering spectrum

(76) Inventors: Murat Canpolat, Plantation, FL (US); Umit Demir, Plantation, FL (US)

Correspondence Address: Murat Canpolat, 911 NW 85th Terrace Apt. 1305, Plantation, FL, 33324, US

(21) Appl. No.: 10/101,934

(22) Filed: Mar. 21, 2002

Related U.S. Application Data
(60) Provisional application No. 60/278,363, filed on Mar. 26, 2001.

(51) Int. Cl. A61B 6/00

(52) U.S. Cl. 600/473

(57) ABSTRACT

This invention is a real-time non-invasive cancer diagnosis method, which is based on detecting morphological alteration of cancer cells in-vivo using elastic light scattering spectrum. An apparatus and method is developed for recording back-scattered light in a small angle range limited by numerical apparatus of a single fiber optical probe. The same optical probe is used to illuminate a tissue and to collect the light scattered back from the tissue. To test our system, we used five Balb/c mice that were injected EMT-6 mammary cells in breast region. We took spectra of the light scattered back from tumors and breast epithelial tissue on the mice. Average size of scatterers in tissue is estimated by fitting spectra of the back-reflected light to Mie theory.

### BACKGROUND OF THE INVENTION

Non-invasive and real time cancer diagnosis would increase survival rate of the patients, reduce medical cost, and help doctors during surgery to define cancerous region of tissue. There are two known methods of non-invasive cancer diagnosis as diffuse reflectance spectroscopy and fluorescence spectroscopy, both of which are based on detection of biochemical and physical variation of the diseased tissue.

Diffuse reflectance spectroscopy has been used in optical biopsy to differentiate diseased tissue from normal tissue. Diffusion approximation estimates optical coefficients of tissue such as reduced scattering and absorption coefficients in the optical biopsy. In the non-invasive diagnosis, the diffusion approximation is used to analyze diffuse back-reflected light with estimated optical coefficients of tissue, which are correlated to physical structure and chemical composition of the tissue. The diffusion approximation works in tissue diagnosis, if the distance between source and detector fibers is at least 3-4 millimeters. Direction of photon scattering must be randomized before photons reach the detector so that the diffusion approximation can be valid. The diffusion approximation is also used to measure average particle size in dense suspensions in frequency domain photon migration and other photon diffusion based techniques. The disadvantage of the photon diffusion based techniques is that the volume sampled must be large enough to hold diffusion approximation, i. e., ˜1 cm³.

Fluorescence spectroscopy is the other non-invasive cancer diagnosis method to differentiate neo-plastic tissue from normal tissue. Here, ultraviolet laser light illuminates the interested tissue area where fluorescence spectra are detected. Fluorescence spectrum of the diseased area is different from that of normal tissue due to biochemical and physical variation of the diseased tissue. Since tissue structure also depends on patient's age, fluorescence spectroscopy results in different outcomes for different ages, which reduces sensitivity and specificity of the method.

The non-invasive cancer diagnosis methods mentioned above are based on biochemical and physical variation of the diseased tissue. Other than these methods, there are on-going research studies in the area of non-invasive cancer diagnosis based on morphological alteration of cell structure for cancer cells, because nuclei of cancerous cells are significantly larger than nuclei of normal cells for many cancer types. Target of these research studies is to estimate average size of scatterers such as nuclei, mitochondria, and other organelles of cells, etc., non-invasively through an optical system

One way of getting information about average size of scatterers is detecting single scattered photons. Direction of scattered photons depends on size, index of refraction, and shape of the particles for a single wavelength of incident light. Single scattering of collimated light is used to measure size of cells and sub-cellular structures in suspension. However, concentration of the scatterers in suspension must be low so that information obtained from only angular distribution of single scattered photons can be analyzed. Three-dimensional computation shows that small organelles play a significant role in light scattering from cells. A. Dunn and R. Richards-Kortum, “Three-Dimensional Computation of Light Scattering From Cells”, IEEE Journal of Selected Topics in Quantum Electronics, 2, 898 (1996). At this study, scattering pattern of light versus scattering angle for a single wavelength of light is calculated. Scattering of light from nucleus only, cell with mitochondria, and cell with melanin has different patterns. The results show that wavy pattern of scattered light disappears if light is scattered by not only nucleus but also by other organelles. Perelman et al. have detected back-scattered light from mono-layer epithelial and T84 cells to estimate average size distribution and concentration of nuclei using an optical probe with one source and six detector fibers. L. T. Perelman et al., “Detection of Periodic Fine Structure in Reflectance from Biological Tissue: A New Technique for Measuring Nuclear Size Distribution”, Phys. Rev. Lett. 80, 627 (1998). Canpolat et al. used single fiber optical probe to estimate size of mono-dispersed scatterers in a turbid medium in-vitro. M. Canpolat et al., “Particle size analysis of turbid media with a single optical fiber in contact with the medium to deliver and detect white light”, Applied Optics 40, 3792 (2001).

The non-invasive diagnosis technique we developed detects morphological alteration of cancerous cells using elastic light scattering.

### SUMMARY OF THE INVENTION

The present invention involves detection of cancerous cells using elastic scattering signal. Back-scattered light can be classified as ballistic and diffuse reflected light. If collected light in back-scattering geometry is scattered off particles once, it carries information about size, and shape of the particles, and their relative index of refraction compared to the surrounding medium.

Light scatterers in a medium due to heterogeneity in index of refraction of the medium. Light scattering in a tissue occurs at cell membrane, nuclei and other organelles in a cell. Index of refraction for cell membrane is greater then index of refraction for extra-cellular liquid, which causes scattering of light at the cell membrane. Light is also scattered inside a cell by nucleus, mitochondria and other organelles due to difference in index of refraction between intracellular compartments and surrounding cytoplasm.

Nucleus size is larger in cancerous cells then that in normal cells, which causes different angular distribution of the scattered light. In our technique, this difference is used to distinguish cancerous cells from normal cells. We assume that relative index of refraction (index of refraction for cell/index of refraction for extra-cellular liquid) is equal in normal and cancerous tissues. Therefore, changes in angular distribution of the scattered light are only dependent on variations in size of the scatterers in cells.

In our system, a broadband light source is used to illuminate tissue surface. Angular distribution of back-scattered light is a function of wavelength of the incident light. Back-scattering light from mono-dispersed particles collected in a small angle range has a pattern of oscillations as a function of wavelength. Oscillations on the pattern become clearer, when back-scattering light is collected in a narrower angle range. As the angular range gets larger, patterns start to disappear due to averaging the back-reflected light over a wide angular scattering range and over multiple scatterings. The important fact is that spectrum of the back-scattering light has oscillation patterns if scatterers are mono-dispersed. Mie theory shows that if scatterers have a size distribution, in other words if scatterers are poly-dispersed, then the oscillation patterns disappear. Therefore, according to Mie theory, there should not be oscillation patterns on the spectra of the back-reflected light from tissue, because light scatters from intracellular compartments with different sizes in the tissue. We do not observe any oscillations in tissue spectra according to our experimental results, which is consistent with the study referenced in. Because of the poly-dispersed nature of the scatterers in tissue, we can estimate the average scatterers' size. We do this by ‘fitting’ the spectra of back-reflected light to Mie theory, where the average and the standard deviation of scatterers' size are our fit parameters.

To test our system in vivo, EMT6 tumors were developed on the breast region of five Bulb\C mice. We took spectra inside tumor and on breast epithelial tissue of each mouse.

Nearly 90% of cancer types initiate at epithelial tissue layer. Since in our system single fiber optical probe detects scattered light at epithelium tissue, it may be possible to detect cancer at its early stage, before it becomes invasive.

### BRIEF DESCRIPTION OF THE DRAWINGS

FIG. 1 is a schematic diagram of the system.

FIG. 2 is a graph of the elastic scattering spectrum of polystyrene micro spheres with diameter of 2 microns.

FIG. 3 is a graph of the light spectra scattered back from epithelial tissue, from tumor surface, and from inside of the tumor. FIG. 3(a) is the graph for the first mouse, (b) for the second mouse, and (c) for the third mouse.

FIG. 4 is a graph of the light spectra scattered from epithelial tissue and from inside of the tumor. FIG. 4(a) is the graph for the fourth mouse, and (b) for the fifth mouse.

### DETAILED DESCRIPTION OF THE INVENTION

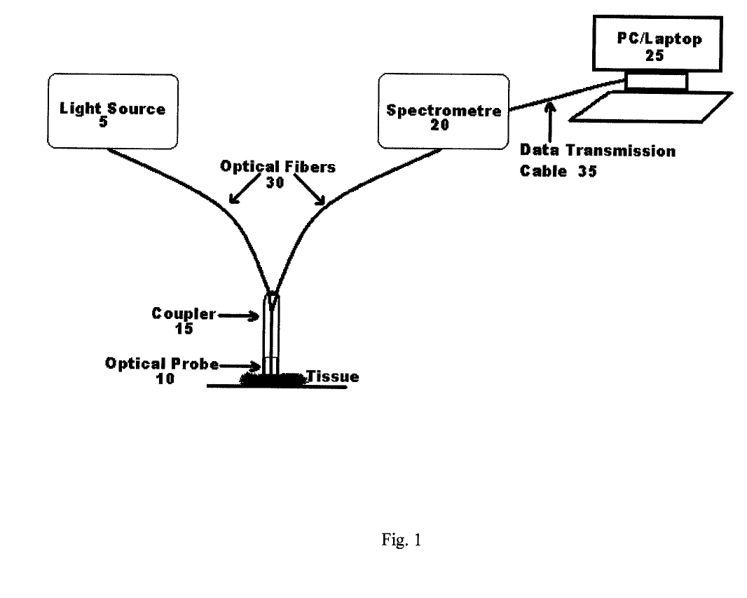

Schematic diagram of the system used in accordance with the present invention consists of a broadband light source 5, a single fiber optical probe 10, a coupler of 1X2 with ratio of 50% 15, a spectrometer 20, a computer 25, optical fibers 30, and a data transmission cable 35 seen in FIG. 1. There is a CCD device detecting light in the spectrometer.

As seen in FIG. 1, the probe consists of only one optical fiber, which delivers light to tissue and collects the light scattered back from the tissue. The optical probe has core and clad diameters of 100 μm and 140 μm respectively. Numerical aperture of the fiber in the optical probe is 0.29. Using single fiber optical probe with a small core diameter and a small numerical aperture enables us to collect mostly single scattered light. The spectrometer (Ocean Optics, FL) measures the spectrum of the light scattered back from the target tissue, and it is connected to a computer, through which users of the system can view the measured spectrum in real-time, and analyze the measurements.

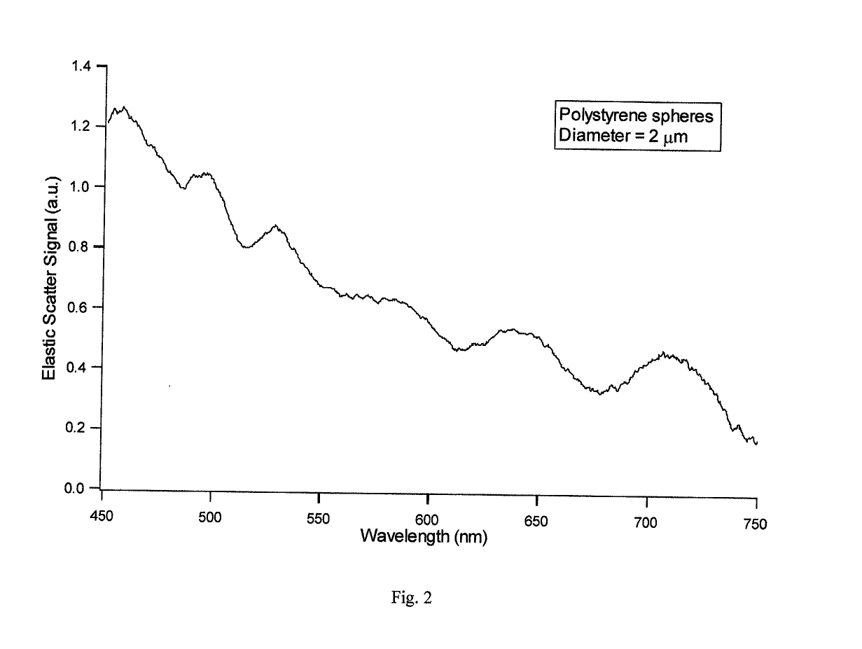

Average size of the scatterers in a turbid medium is estimated by fitting spectra of back-reflected light to Mie theory. To test our system before our in-vivo experiments, we use mono-dispersed polystyrene particles, and their diameter is 2 μm, according to the manufacturer specifications (Duke Scientific Corporation, Palo Alto, Calif.). The spectrum for aqueous solution of polystyrene particles with diameter of 2 μm is seen in FIG. 2. Oscillations on the spectra are seen clearly in the figure. We estimate size of the polystyrene particles by fitting the spectrum in the FIG. 2 to Mie theory, and according to the fit results, estimated diameter length of the micro spheres is 1.634 μm, which is 19.3% different then its actual value.

The initial step of our in-vivo experiments was to inject EMT-6 mammary adenocarcinoma cells in breast region of five Balb/c mice. After ten days, average size of the tumors reached to 125 millimeter cubed. First, each mouse was put into sleep then sacrificed by a biologist in Rumbaugh Goodwin Institute for Cancer Research. Grown tumor and normal breast tissue were removed from the mice. Right after the biopsy, we took 5-10 spectra on each sample in 20 minutes. Before taking spectra from each sample, tip of the probe was cleaned and then a spectrum from polystyrene particles is taken to check probe performance and to calibrate the software set-up as necessary.

Spectra taken from polystyrene particles and tissue samples are corrected for wavelength dependence of the system components and specular reflection. The corrected spectrum is

$${I}_{cor} = \frac{{I}_{mes} - {I}_{beck}}{{I}_{spectralon} - {I}_{beck}} ,$$

Where Iₘₑₛ is a scattering spectrum of polystyrene solution or a tissue sample, I_{beck} is a spectrum taken from distillated water in a black container, and I_{spectralon} is spectrum of spectralon (Ocean Optics, FL) in water. From this point further, we will call “corrected spectrum”, I_{COr,}as “spectrum”.

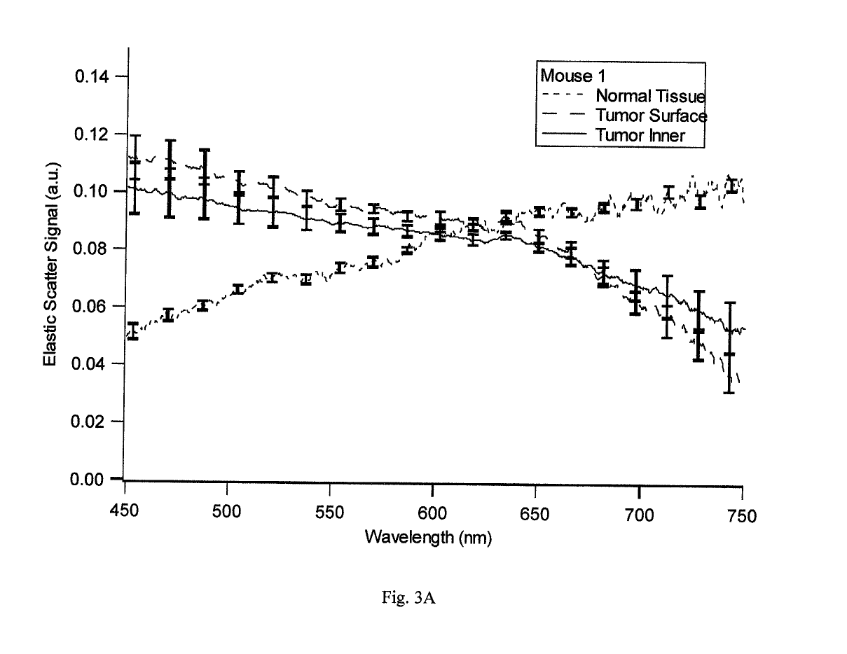

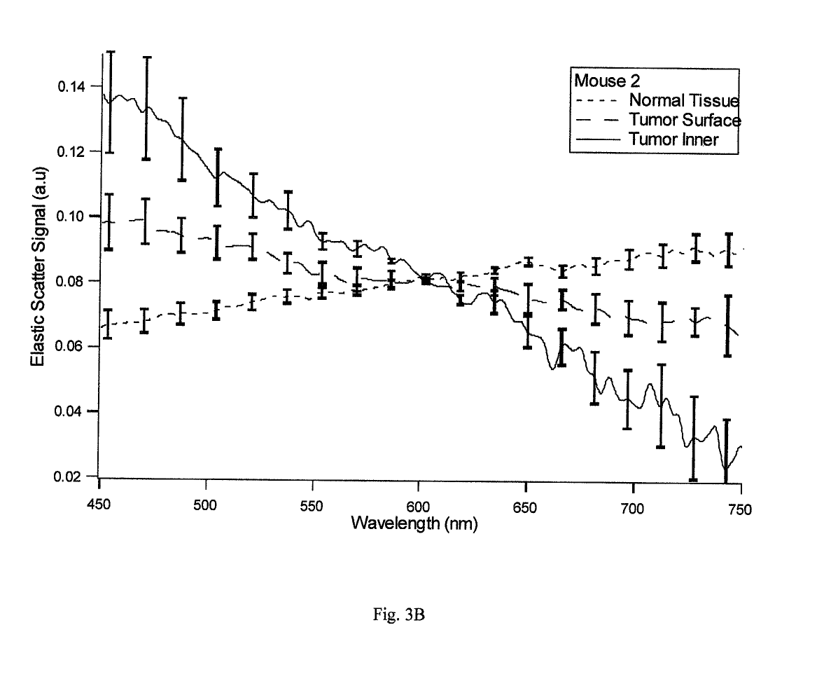

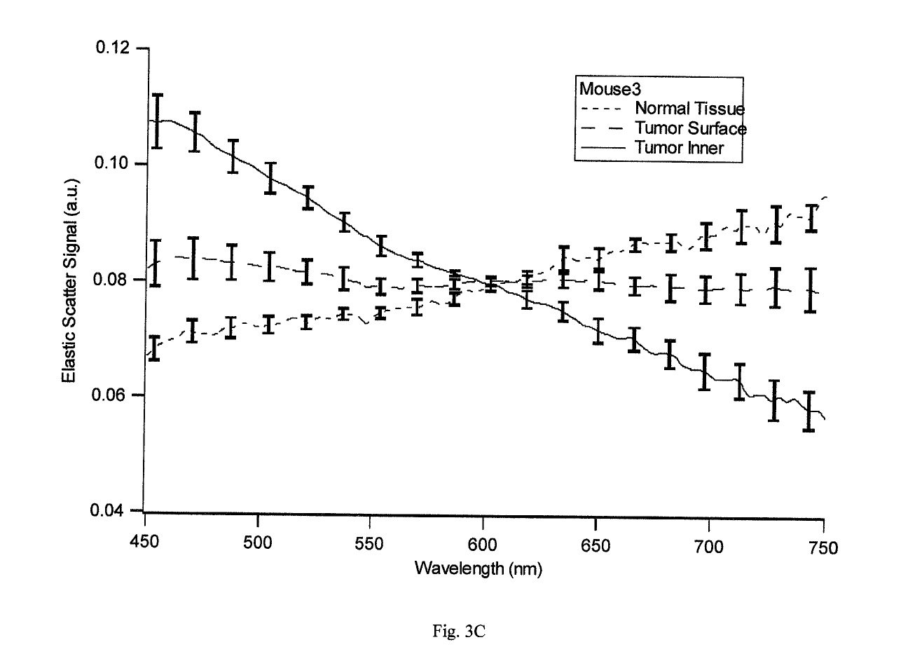

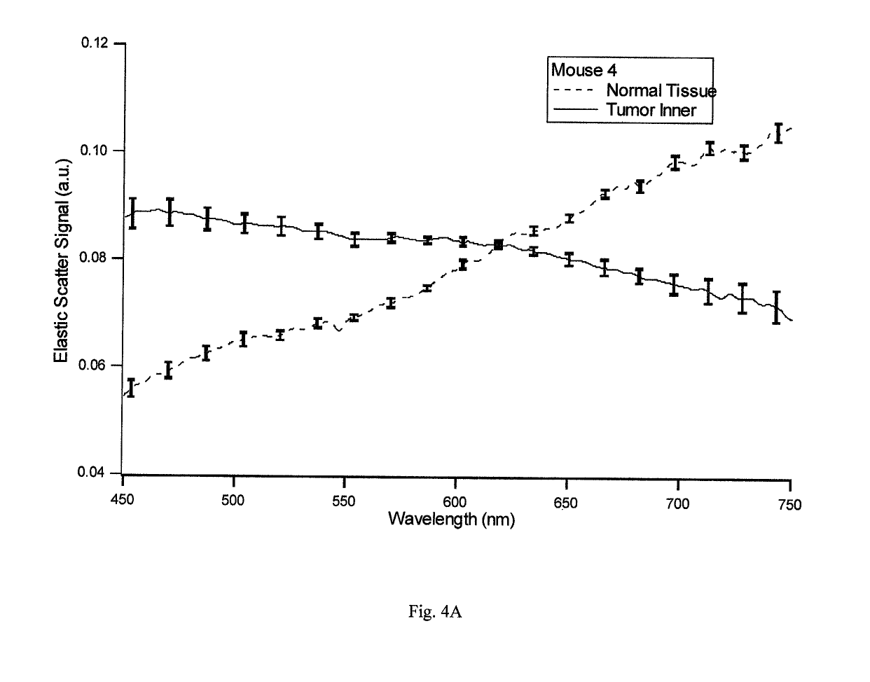

We took spectra from three different locations of mouse tissue. The first spectrum was taken from normal breast epithelial tissue, the second one taken from tumor surface, and the third one taken from inside of the tumor. Spectra between wavelengths of 450 nm and 750 nm were normalized to remove any intensity variation in measurement. Spectra from the first three mice are seen in FIG. 3. Spectra in FIG. 3 are for normal breast epithelial tissue, tumor surface, and inside tumors. In FIG. 3, there are two important observations to be recognized:

The first observation is that there are no oscillations on the spectra of the normal and cancerous cells. This proves the poly-dispersed nature of the scatterers' size in tissue. Therefore, we assume that scatterers' size have a Gaussian distribution in tissue. Two free parameters are used to fit the spectra to Mie theory. Distribution of the scattered light in the angular range of 163-180 is numerically calculated using the integral

$$( π / {k}^{2} ) {\int }_{160}^{180} ( {\vert {S}_{1} ( ϑ ) \vert}^{2} + {\vert {S}_{2} ( ϑ ) \vert}^{2} ) sin ϑ ⅆ ϑ .$$

Where k is wave number of light, θ is scattering angle, and S₁, S₂ are scattering amplitudes in Mie theory. The two free fit parameters are the average and the standard deviation of scatterers' size. Fitting algorithm outputs average size of the scatterer distribution in breast epithelial tissue and tumors. Average size of the scatterers in the cancerous cells is 1.975 μm, which is larger then average size of scatterers in the normal breast epithelial tissue, which is 0.648 μm. Here we know from in-vitro experiments that error in the calculation of the scatterer's size is less then 20%.

In biological cells, where there are many organelles smaller then the nucleus, the average scatterer size ranges from 0.4 to 2.0 μm. J. Mourant, et al., “Mechanisms of light scattering from biological cells relevant to noninvasive optical-tissue diagnostics.” Appl. Opt. 37, 3586-3593 (1998). The same study shows that the nucleus contributes mostly to low angle scattering while small organelles contribute to high angle scattering. We have measured average size of the scatterers in tissue in-vivo and estimated the average scatterer size in normal and cancerous cells. Our numbers are consistent with the numbers referred here from the reference (See, J. Mourant et al., Mechanism of light).

Perelman et al measured average size of the nuclei in normal and cancerous cells using light scattering technique. L. T. Perelman et al “Observation of Periodic Fine Structure in Reflectance from Biological Tissue: A New Technique for Measuring Nuclear Size Distribution”, Phys. Rev. Lett. 80, 627 (1998). Average size of epithelium and T84 tumor cells are 6.2 μm and 10.1 μm respectively. Measured values of the normal and cancer nuclei by a light microscopy are 6 μm and 10.2 μm. Our measured values for the average scatterer size of breast epithelial tissue and tumor are smaller then the average nucleus size because light is scattered by not only nuclei but also by other organelles in cells.

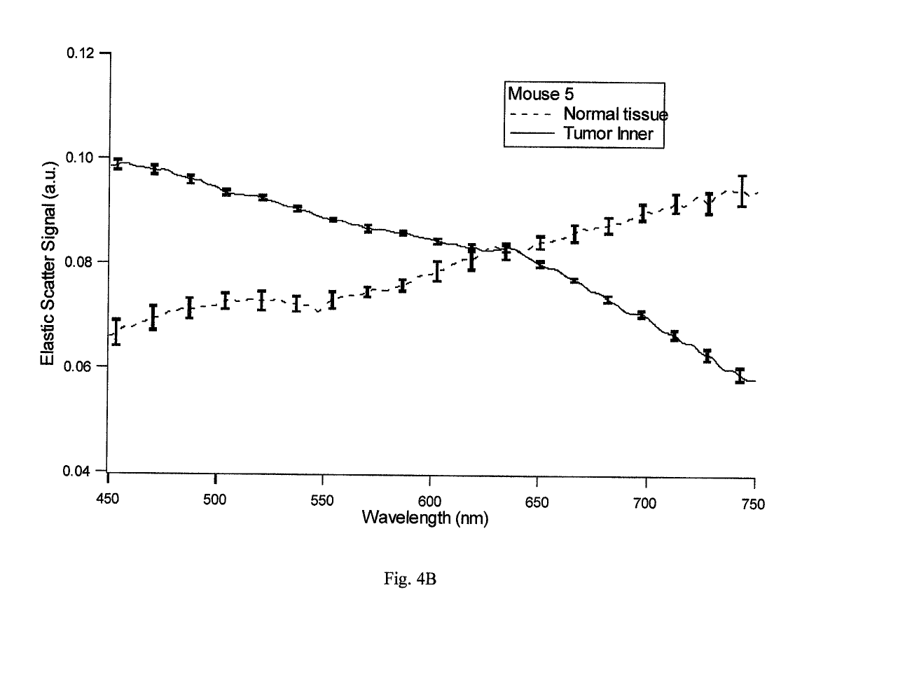

The second important observation in our experimental results is that slope of the spectra is positive for epithelium tissue and negative for cells inside the tumors, as it can be seen in FIG. 3b and FIG. 3c. Slope of the spectra taken from tumor surface is nearly zero since light scatters from both normal cells in a membrane and the tumor encapsulated by the membrane. The membrane itself may also have cancerous cells. Therefore, spectra of the tumor surface are average of spectra of normal and cancerous cells. Spectra taken from the tumor surface and inside of the tumor for the first mouse are similar as seen in FIG. 3a, because membrane on the tumor surface of the first mouse was broken during experimentation. Spectra of the epithelium tissue and inside of the tumor for the last two mice are in FIG. 4 and consistent with the results in FIG. 3.

There are very important similarities between our experimental results and the theoretical model of light scattering in normal and pre-cancerous cervical cells. R. Drezek et al., “A Pulsed Finite-Difference Time-Domain Method for Calculating Light Scattering from Biological Cells Over Broad Wavelength Ranges”, Optics Express 6, 147 (2000). At this study, Drezek et al. modeled heterogeneous normal and pre-cancerous cervical cells with diameter of 9 μm. Light scattering from the cells was calculated by a pulsed finite-difference time-domain method. In the simulation, broad band light in the range of 600 nm-1000 nm was used. Intensity of the scattered light was integrated as a function of wavelength for different angular ranges. The difference between the integrated intensities of normal and pre-cancerous cells is the most dominant for the angular range of 160-180 degrees, where intensity of scattered light increases with wavelength for normal cells and almost does not change in the whole wavelength range for dysplasia. According to our experimental results, intensity of the back-scattered light increases with wavelength for normal cells as in Drezek's simulation. Also, spectra of the tumor surface and the epithelial tissue, as seen in FIG. 3a, FIG. 3b, and FIG. 3c, are similar to the intensity patterns of light scattered by normal and dysplastic cells in the simulation (See, R. Drezek et al) . As indicated before, tumor is capsulated by a membrane where most of the time there are both cancerous and normal cells. Therefore the light scattering from the tumor capsule has a similar pattern to the light pattern of the dysplastic cells. In the tumor we took spectra from adeniocarcionoma cells, where intensity of the scattered light decreases with the wavelength In Drezek's simulation, slope for the calculated spectra of normal cells is positive, and it approaches to zero, as the cell morphology becomes dyplastic. Results of these simulations are consistent with our experimental results, as shown in FIG. 3 and FIG. 4, where slope for the measured spectra of normal epithelial tissue is positive, slope for the measured spectra of tumor surface is nearly zero, and slope of the spectra measured inside the tumor is negative.

What is claimed is:

1. A method of detecting morphological alteration of cancer cells at tissue surface comprising:
Using a broadband light source to illuminate tissue surface;
Collecting scattered radiation from the tissue surface;
Analyzing the collected light to estimate average size for a Gaussian distribution of scatterers in tissue using the spectrum of the collected light;
Using a single optical fiber probe to radiate tissue and collect back-scattered light from the tissue by the same probe;
Detecting non-diffuse back-reflected light from tissue surface;

2. The method of claim 1, wherein the optical probe has a diameter in the range from 1 μm to 600 μm.

3. The method of claim 1 further comprising directing radiation onto the tissue using a single fiber optical probe.

4. The method of claim 1 further comprising collecting the radiation from the tissue with the same single fiber optic probe, which is used to illuminate tissue surface.

5. The method of claim 1 further comprising determining an average scatterer size within the region of interest. The method of claim 1 flirter comprising measuring intensity of the single back-scattered light in the angle ranges of 120-180 degree as a function of wavelength.

6. A method of optically measuring tissue comprising the steps of:
directing incident radiation onto tissue;
collecting scattered radiation from the tissue; and
fitting scattered radiation to the Mie theory to measure average size of the scatterers in tissue.

7. The method of claim 6 further comprising directing radiation onto the tissue using a single fiber optical probe.

8. The method of claim 6 further comprising collecting the radiation from the tissue with a single fiber optical probe.

9. The method of claim 6 further comprising differentiating normal, and cancerous tissue.

10. The method of claim 6 further comprising determining an average size of scatterers within the region of interest.

11. The method of claim 6 further comprising collecting radiation with single fiber optical probe by;
touching the probe to outer surface of the tissue;
inserting the probe inside an endoscope;
inserting the probe inside a biopsy needle.

12. An apparatus for optically measuring tissue comprising:
a radiation source that illuminates a region of interest in tissue;
a single fiber optical probe that collects scattered radiation from the tissue;
a detector system that counts the collected scattered radiation; and
a data processor that uses spectrum from water in a dark container, and spectrum from spectralon in water to eliminate the spectral distribution of light source and to remove any back refraction from the system.

13. The apparatus of claim 12 further comprising a broadband light source that generates light in a range of 300-1200 nm.

14. The apparatus of claim 12 further comprising a fiber optic probe that touches to tissue fits, inside an endoscope and fits inside a biopsy needle.

15. The apparatus of claim 12 flirter comprising a fiber optic probe that collects the light in a collection angle between in the range of 120 -180 degrees.
